(19) United States
(12) Patent Application Publication
(10) Pub. No.: US 2015/0126903 A1
(43) Pub. Date: May 7, 2015

(54) integrated punch biopsy device

(71) Applicant: The Johns Hopkins University, Baltimore, MD (US)

(72) Inventors: Timothy Wang, Baltimore, MD (US); Sewon Kang, Baltimore, MD (US); Nishant Genesh Kumar, Columbia, MD (US); Makoto Tanigawa, Baltimore, MD (US); Sebastian Kwon, Okemos, MI (US); Abhilash Guduru, Raleigh, NC (US); Kallum Cheng, Jamaica, NY (US); Tomas Gaigalas, Gaithersburg, MD (US); Bofeng Zhang, Novi, MI (US)

(21) Appl. No.: 14/398,435

(22) PCT Filed: May 3, 2013
(86) PCT No.: PCT/US2013/039552

§ 371 Date: Oct. 31, 2014

Related U.S. Application Data
(60) Provisional application No. 61/642,158, filed on May 3, 2012.

(51) Int. Cl. A61B 10/02

(52) U.S. Cl. CPC A61B 10/0266; A61B 2010/0208
USPC 600/567

(57) ABSTRACT

A punch biopsy tool includes a punch cutting element which includes an inner surface, an outer surface, and a cutting edge along a bottom edge, and which defines an aperture between the inner surface and the outer surface. The punch biopsy tool also includes a flexible cutting element which is disposed along the inner surface of the punch cutting element and is configured to be pulled through the aperture.

### BACKGROUND

1. Field of Invention

The field of the currently claimed embodiments of this invention relates to medical tools, and more particularly to punch biopsy tools.

2. Discussion of Related Art

When faced with an abnormal skin lesion, physicians are often required to perform a biopsy to obtain a sample of the tissue. Types of skin biopsies include shave, excisional, and punch, and the samples provided by each differ in size and depth. Punch biopsies are the primary technique used to acquire full-thickness specimens because they obtain the epidermis, the dermis and the subcutaneous tissue. Punch biopsies, however, are technique-dependent and require multiple surgical instruments to perform. Two aspects of the procedure, namely the depth of the punch (sample thickness) and the mechanical handling of the specimen can particularly affect the quality of the sample as inadequate sample depth or mechanically induced crush artifact can prevent pathologists from making accurate diagnoses.

The current procedure can be divided into three phases. There is the ‘punch’ phase during which the punch circular blade enters the skin. This is followed by the ‘excision’ phase where the sample is transected or “cut” at its base and finally by the ‘closure’ phase where the wound is sutured. For the punch phase, damage to underlying artery or nerve could be done if the depth of the punch is not regulated. In the excision phase, integrity of the skin sample could be compromised if the sample is severed traumatically. A damaged sample undermines the diagnosis done by the pathologist. The closure phase is the most time consuming phase of the procedure. Further, the current clinical set up requires many instruments all of which take time to sterilize using an autoclave.

### SUMMARY

According to an embodiment of the present invention, a punch biopsy tool is disclosed. The punch biopsy tool can include a punch cutting element which includes an inner surface, an outer surface, and a cutting edge along a bottom edge, and which defines an aperture between the inner surface and the outer surface. The punch biopsy tool can also include a flexible cutting element which can be disposed along the inner surface of the punch cutting element and can be configured to be pulled through the aperture.

According to another embodiment of the present invention, a biological tissue removal tool is disclosed. The tissue removal tool can include a hollow cylindrical shaft wherein the apex is threaded on the outer surface and wherein a first groove runs the length of the inner surface. The tissue removal tool can additionally include a cylindrical blade attached to the base of the shaft wherein the cutting edge is the base of the blade and wherein a second groove runs the length of the outer surface and aligns with the first groove. A hole through the blade can be located in the second groove. The tissue removal tool can also include a wire configured as a lasso on the inside of the blade wherein the wire is flush with the inner surface of the blade and wherein the wire exits through the hole and wherein the wire runs along the second groove and continues along the first groove in the shaft. The tissue removal tool can further include a removable handle to which the wire is attached, and a sleeve wherein threads on the inner surface are complementary with threads on the shaft, and the base of the shaft may be extended beyond the base of the sleeve.

### BRIEF DESCRIPTION OF THE DRAWINGS

Further objectives and advantages will become apparent from a consideration of the description, drawings, and examples.

FIG. 1 shows an embodiment of a punch biopsy tool.

FIG. 2 shows another embodiment of a punch biopsy tool.

FIGS. 3A-3E show an embodiment of a punch cutting element.

FIGS. 4A-C show an embodiment of an upwardly extending support structure.

FIG. 5 shows an example process for collecting a biopsy sample using a punch biopsy tool.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

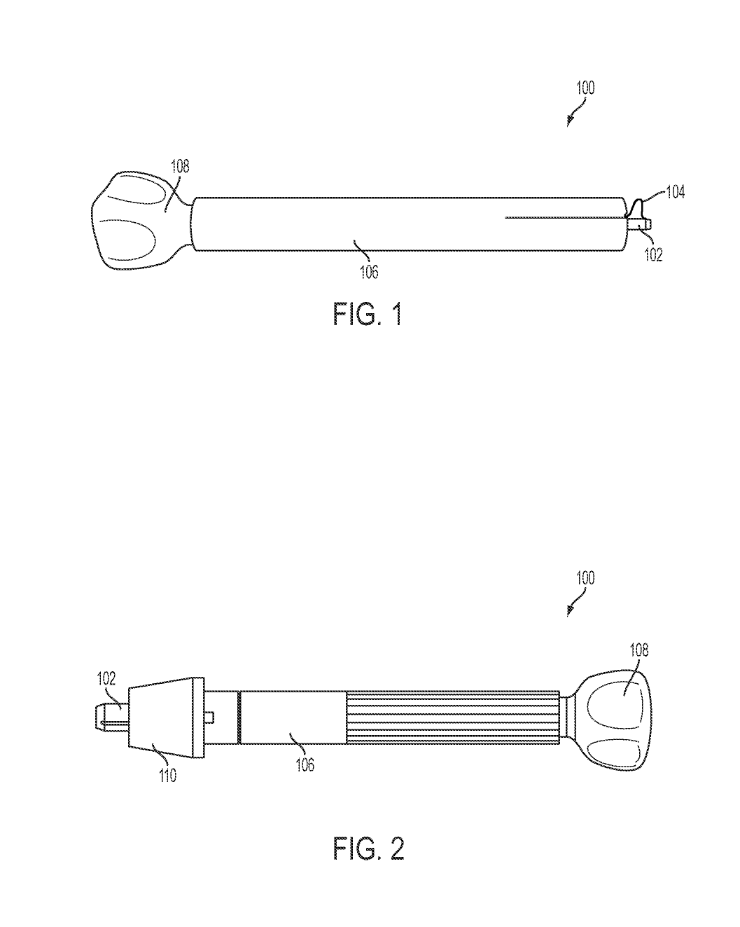

FIG. 1 shows an embodiment of a punch biopsy tool 100. Punch biopsy tool 100 can include a punch cutting element 102. Punch cutting element 102 can be constructed of any suitable material, for example a metal such as surgical stainless steel or type 304 stainless steel. Alternatives could include, but are not limited to, ceramic materials used for cutting blades. Punch biopsy tool 100 can also include a flexible cutting element 104. Flexible cutting element 104 can be in any configuration, and can be constructed of any suitable material. For example, flexible cutting element 104 can be any type of cutting wire, for example a diamond-coated wire, and can be of any size as desired, for example 36-gauge. Punch biopsy tool 100 can also include an upwardly extending support structure 106, a handle 108. FIG. 2 shows another embodiment of a punch biopsy tool 100 that further includes a sleeve 110.

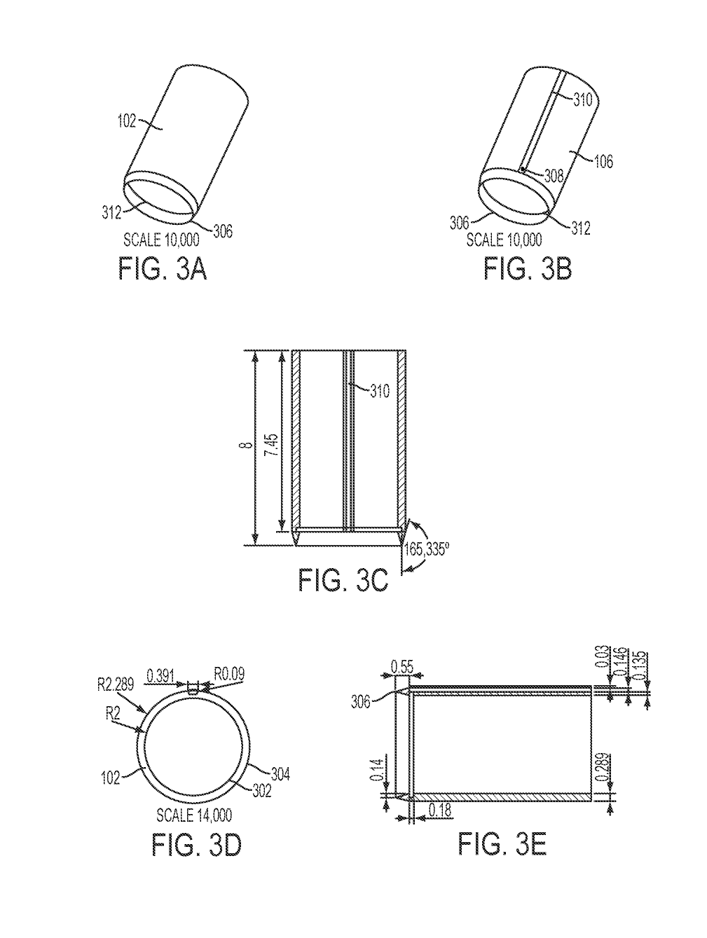

FIGS. 3A-E show an embodiment of a punch cutting element 102. Punch cutting element 102 can have a substantially circular cross section with an inner surface 302 and an outer surface 304. In other embodiments, punch cutting element 102 can be any shape as desired. For example the cross section of punch cutting element 102 can be oval or rectangular in shape, or have an irregular shape, as desired. Punch cutting element 102 can include a cutting edge 306, which can be disposed along a bottom edge of punch cutting element 102. The diameter of inner surface 302 can be smaller than the diameter of cutting edge 306, which can in some embodiments assist with the removal of a sample.

Still referring to FIGS. 3A-E, punch cutting element 102 can define an aperture 308 between inner surface 302 and outer surface 304. Aperture 308 can be located, for example, above cutting edge 306. In other embodiments, aperture 308 can be located in any other location on punch cutting element 102 as desired. Punch cutting element 102 can include a groove 310 in outer surface 304. Groove 310 can run substantially vertically down outer surface 310. Punch cutting element 102 can also include a channel 312 in inner surface 302. Channel 312 can run horizontally along inner surface 302 around the circumference of punch cutting element 102. In other embodiments groove 310 and channel 312 can be in any location or orientation as desired. Aperture 308 can be located in such a way that it intersects both groove 310 and channel 312.

Still referring to FIGS. 3A-E, flexible cutting element 104 can be configured such that it can be disposed along groove 310 and channel 102, and can be inserted through aperture 308. Groove 310 can be configured in such a way that it acts as a guide for the movement of flexible cutting element 104 along outer surface 304. Channel 312 can be configured in such a way that when flexible cutting element 104 is disposed along channel 312, flexible cutting element 104 is flush with inner surface 302. In some embodiments, flexible cutting element 104 can be configured so that a portion of flexible cutting element 104 can be tied, woven, twisted, or otherwise configured as a loop. In this way a portion of flexible cutting element 104 can run along groove 310, and another portion of flexible cutting element 104 can be inserted through aperture 308, configured as a loop, and disposed along channel 312. Flexible cutting element 104 can be configured so that as flexible cutting element 104 is pulled outward through aperture 308, the diameter of the loop formed by a portion of flexible cutting element 104 is decreased.

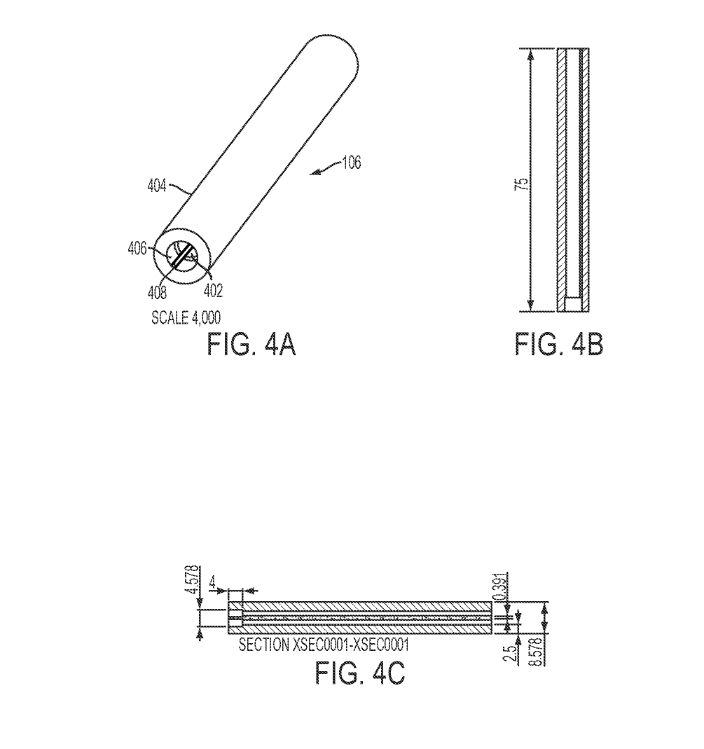

FIGS. 4A-C show an embodiment of an upwardly extending support structure 106. Upwardly extending support structure 106 can be constructed of any material, as desired, for example any type of metal or plastic. Upwardly extending support structure 106 can be configured to be gripped by a hand, manipulated by another tool, or used in any other way as desired. Upwardly extending support structure 106 can include an inner surface 402 and an outer surface 404. Inner surface 402 can include a pocket 406 sized and shaped to receive punch cutting element 102. Punch cutting element 102 can be removable from pocket 406 so that punch cutting element 102 can be replaced, sterilized, or otherwise cleaned. Inner surface 402 can also include a groove 408 which can correspond in size and location to groove 310 and can act as a guide for the movement of flexible cutting element 104 along inner surface 402.

In some embodiments, a portion of outer surface 404 can be threaded or otherwise configured to accept a sleeve 110. Sleeve 110 can be positioned in any orientation along outer surface 404. In this way, the position of sleeve 110 can determine how much of punch cutting element 102 extends from the bottom of punch biopsy tool 100.

In some embodiments, punch biopsy tool 100 can include handle 108. Handle 108 can be rotatably or removably attached to both flexible cutting element 104 and upwardly extending support structure 106. Handle 108 can be attached to flexible cutting element 104 and inserted into upwardly extending support structure 106 in such a way that rotating or removing handle 108 causes flexible cutting element 104 to move upwardly along grooves 408 and 310 and outwardly through aperture 308. In this way, rotating or removing handle 108 can cause the diameter of the loop formed by a portion of flexible cutting element 104 to decrease.

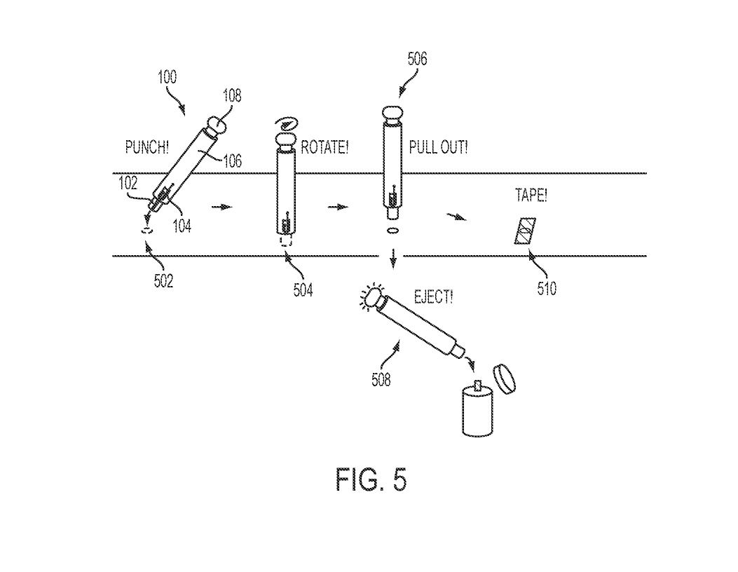

FIG. 5 shows an example procedure for using punch biopsy tool to obtain a biopsy sample. At step 502, punch biopsy tool 100 can be inserted into a portion of a patient's skin, while flexible cutting element 104 remains flush with inner surface 302. At step 504, handle 108 can be rotated, removed, or otherwise manipulated. This can cause the diameter of the loop formed by a portion of flexible cutting element 104 to be decreased, and can eventually result in the removal of flexible cutting element 104 from the interior portion of punch cutting element 102. This can cause the bottom of the sample to be severed from the rest of the patient's skin. At step 506, punch biopsy tool 100 can be removed from the patient's skin, with the sample retained within punch cutting element 102 by the force of friction. At step 508 the sample can be ejected from punch biopsy tool 100, and at step 510 the defect left by the removal of the sample can be treated, for example by the application of a tape or bandage.

### EXAMPLES

The following examples are provided by way of illustration only and are not intended to be limiting.

Punch biopsy tool 100 can alter the design of the current punch tool, significantly decreasing the need for additional instruments, reducing cost and improving safety. The device can efficiently and safely transect and retrieve the skin sample. Closure of the defect can also be addressed.

In some embodiments, the punch tool can be modified to sever the bottom of the sample and to regulate the depth of the sample. A wire lasso can sit flush within a circumferential canal machined into the inner surface of the circular blade. The wire can exit the inner aspect and courses up the length of the shaft in a similar canal cut into the outer aspect of the shaft to a removable handle. Because the wire lasso can be incorporated into the blade, cutting the bottom of the skin sample does not damage the integrity of the sample. The depth regulation mechanism can be a sleeve that can attach to the handle of the biopsy punch tool which enables adjustment of the blade depth based on predetermined increments. An adhesive closure strip can eliminate the need for suturing. If necessary, attachment of the strip can be enhanced by the use of liquid adhesive contained within our kit ensuring that the defect remains closed for sufficient time to heal.

The device can include four main components: a plastic shaft or support structure, a punch cutting element which can as an example be a cylindrical blade, a flexible cutting element which can as an example be a 150 micron diamond coated wire and a rotatable or removable handle. There can also be an attachable sleeve that can be used to regulate the depth of the punch. The plastic shaft can be a hollow cylinder with the dimensions shown in FIG. 4. At the base of the shaft can be located a pocket in which the blade can be attached. A groove can be present along the inside of the plastic shaft that can be continuous with a similar groove on the outside surface of the proposed blade (FIG. 3). The wire can form a lasso on the inside of the blade so as to be flush with the inner surface then exits through an opening in the blade. The wire can then run upwards along the outer groove of the blade that it continues into the shaft and finally attaches to the base of a rotatable handle. The blade can be made of type 304 stainless steel with the dimensions (FIG. 3). The cutting edge can be located at the base of the blade. Also near the base along the inside of the blade can be a circular groove in which the wire can rest. A circular hole within the groove can allow the wire to pass through from the inside of the blade to the outside. The 150 micron diamond coated wire that can run up into the plastic shaft can be attached to the base of a rotatable handle. The attachable sleeve can have threads on the inside that can be complementary with threads on the shaft. This can enable depth regulation by acting as a movable hub that regulates how deep the blade can pierce the skin. It can be screwed on or off the shaft with a circular motion.

The blade can be manufactured from a cylindrical block of type 304 stainless steel. A drill can be used to remove a cylindrical block of steel smaller in diameter to the original block, leaving behind a cylindrical shell. The shell can be refined by boring. Through blade operations, a circular groove can be made along the inside of the shell near the bottom. Finally, a milling machine can be used to drill a small hole through the wall of the blade. The plastic shaft can be made from a cylindrical block of Delrin. A drill can be used to make a hollow cylindrical piece of plastic with a pocket that can hold the blade. The blade can be then glued on to the shaft. Finally, one end of the 150 micron diamond coated wire can be secured to the exterior of the shaft. The other end can enter the interior of the blade through the hole near its base. It can then make a circular loop at the bottom of the blade, flushed with the groove along the inside of the blade. The wire can then exit the interior of the blade through the same hole through which it came in, and retrace itself back up the exterior of the blade, and interior of the shaft. This end of the wire can be secured to the base of the rotatable handle.

One procedure associated with FIG. 5 can proceed as follow. Mark the site of the biopsy on the skin. Rotate the blade into the skin, until the hub meets the skin, to make a circular incision. Rotate the rotatable handle to sever the skin sample from the underlying skin. Remove the device from the skin. The skin sample will be collected within the blade. Use a Q-tip to gently push the skin sample out of the device. Dispose of device. In order to close the punch biopsy site, make use of a steristrip by bringing the edges of the wound together and closing the edges with the steristrip. A medical adhesive may be used to enhance the strength of the closure. In the case of excessive bleeding a suture may be required. Images of the prototype are attached at end of this document.

In some example embodiments, punch biopsy tool 100 can remove a cylindrical skin sample of any depth greater than 2 mm and less than 8 mm with a set a diameter. We expect our device to be manufactured with varying diameters varying from 2 mm to 8 mm to meet the user's needs in terms of different depth and diameters of skin samples. A 150 micron diamond coated wire can be used to excise the bottom of the skin sample. However wires of different diameters can be used depending on the diameter of blade since a thicker skin sample will require a thicker wire. Further the wire can be wound to form a lasso within the blade. The mechanism being used can involve rotating a rotatable handle that narrows the diameter of the wire. A separate cotton tip applicator can be used to eject the skin sample from the blade. However, a mechanism to accomplish this can be integrated into the device to further reduce the number of tools required for the procedure. A Steri-strip® can be used to close the wound. However, based on the needs of the procedure, it is possible to expect to alter the shape of the current product to maximize the effect of closure specifically for a circular incision. To regulate depth, an attachable sleeve can serve as a hub to limit how deep the blade can enter the skin. Alternative methods can be used to regulate the depth, including, for instance, a box cutter type mechanism. A mechanism can be used that can insert the blade into the skin without physically having to cause a circular motion to insert the blade into the skin. For instance, a handle could be incorporated that facilitates this circular motion of the blade into the skin. We have tested our device on a skin substitute used by surgeons to teach residents, as well as on cooked meat (beef) and raw meat (beef, chicken). Most importantly we tested our device on discarded human skin samples from the forehead. Additionally, testing will be done on skin substitute material that current dermatologists and surgeons use to practice procedures on.

Further aspects of punch biopsy tool 100 can include:
A punch biopsy device can include a cylindrical shaft, a diamond coated wire, and a cylindrical metallic blade encased by the aforementioned shaft that is used to enter the skin for a full thickness skin sample that is used for diagnosis.
The device can excise a cylindrical sample of skin of varying depth.
The depth of the skin sample obtained can be regulated.
The skin sample can be separated from the underlying skin without the use of forceps or scissors.
The design of the blade can enable the desired skin sample to be collected within the blade without the need of forceps.
The skin sample can be ejected from the blade with the use of a Q-tip.
A sleeve attached to the cylindrical shaft can regulate depth.
The sleeve can be attached using a screw mechanism that includes complementary threads on the sleeve and the shaft as found in the threads of the cap of commercial plastic bottles.
The sleeve can include a cylindrical shape that is of a wider diameter than the shaft of the punch biopsy device so as to facilitate the additional space needed for the wire to exit up the shaft.
The sleeve can have a solid extension at the base of the sleeve that aims to prevent the underlying skin from entering in between the shaft and the sleeve.
The sleeve facilitates depth regulation in a range of 2 mm to 8 mm using intervals of at least 0.5 mm.
The separation of the skin sample from the underlying skin can be the result of a lasso of diamond coated wire at the base of the metallic blade that reduces in diameter when the wire is pulled upwards.
The diamond coated wire can be pulled upwards with the help of a rotatable handle to which it is attached.
The diamond coated wire can be threaded from outside of the blade to be flush with the inside surface of the blade through a small hole near the base of the blade.
The wire can be threaded through a groove along the inside surface of the blade to form a lasso.
The wire can exit the inside surface of the blade to the outside of the blade through the same hole and travel up the body of the device.
Manually rotating the handle can result in the displacement of said wire which will result in the aforementioned reduction in diameter of said lasso.
The blade can be designed such that the diameter of the cutting edge of the blade is larger than that of the inside surface of said blade allowing for the skin sample to be compressed into the inside of the blade.
The compressed skin sample can exert an increased normal force against the inside surface of the blade thereby increasing the frictional force, allowing the skin sample to be extracted from the skin within the blade.
A Q-tip of diameter smaller than the interior diameter of the shaft can be inserted through an opening at the top of the shaft.
The Q-tip that is inserted through the opening at the top of the shaft can eject the skin sample into a container of the user's choice.

### REFERENCES

Harris, Jeffrey P., George K. Houghton, and Adam B. Harris. Automated Skin Biopsy Device. Patent 2007/0232954 A1. 4 Oct. 2007. [2]Acuderm Inc. “1-8 mm Boxes of 25.” Www.acuderm.com. 2011. Web. 1 Oct. 2011. <http://www.acuderm.com/category_s/2.htm>. [3]“VisiPunch|Biopsy Punch|Medical Lighting|Innovative Medical Solutions|Huot Instruments LLC.” Biopsy Punch|Rotary ElliptiPunch|VisiPunch|DermaPunch|Startrol|Medical Lighting Products|Huot Instruments LLC. Huot Instruments. Web. 29 Sept. 2011. [4]“ElliptiPunch|Biopsy Punch|Medical Lighting|Innovative Medical Solutions|Huot Instruments LLC.” Biopsy Punch|Rotary ElliptiPunch|VisiPunch|DermaPunch|Startrol|Medical Lighting Products|Huot Instruments LLC. Web. 1 Oct. 2011. <http://www.huotinstruments.com/medicalInstrumentsProducts.asp?id=9>. [5]Allen, Michael E. “Sutureless Guided Skin Biopsy System—Google Patents.” Google. October-November 2000. Web. 29 Sept. 2011. [6]Bucalo, Brian D. “Biopsy Instrument with Tissue . . . —Google Patents.” Google. Web. 1 Oct. 2011. <http://www.google.com/patents?id=GlsjAAAAEBAJ>.

American Cancer Society. Cancer Facts & FIGS. 2011. Atlanta: American Cancer Society; 2011.

<http://www. cancer.org/Cancer/CancerCauses/SunandUVExposure/skin-cancer-facts>. [2] “SEER Training: Anatomy of the Skin.” SEER Training: Welcome to SEER Training. Web. 1 Oct. 2011. <http://training.seer.cancer.gov/melanoma/anatomy/>. [3] “Skin Anatomy and Conditions.” Children's Hospital Boston. Web. 1 Oct. 2011. <http://www.childrenshospital.org/az/Site784/mainpageS784P0.html>.

Zuber, Thomas J. “Punch Biopsy of the Skin.” American Family Physician 65.6 (2002).

Punch Biopsy of the Skin. THOMAS J. ZUBER, M.D., Saginaw Cooperative Hospital, Saginaw, Mich.. Am Fam Physician. 2002 Mar 15;65(6):1155-1158.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A punch biopsy tool, comprising:
a punch cutting element which includes an inner surface, an outer surface, and a cutting edge along a bottom edge, and which defines an aperture between the inner surface and the outer surface; and
a flexible cutting element which is disposed along the inner surface of the punch cutting element and is configured to be pulled through the aperture.

2. The cutting tool of claim 1, wherein at least a portion of the flexible cutting element is configured as a loop such that pulling the flexible cutting element through the aperture causes the loop to reduce in diameter.

3. The cutting tool of claim 1, further comprising an upwardly extending support structure removably attached to the punch cutting element.

4. The cutting tool of claim 3, wherein the upwardly extending support structure is configured to be gripped by a hand.

5. The cutting tool of claim 4, further comprising a handle attached to the upwardly extending support structure and the flexible cutting element.

6. The cutting tool of claim 5, wherein the handle is configured to rotate with respect to the upwardly extending support structure.

7. The cutting tool of claim 6, wherein rotating the handle causes the flexible cutting element to be pulled through the aperture.

8. The cutting tool of claim 1, wherein the punch cutting element is substantially cylindrical.

9. The cutting tool of claim 1, wherein the flexible cutting element is a cutting wire.

10. The cutting tool of claim 9, wherein the flexible cutting element is a diamond-coated wire.

11. A biological tissue removal tool, comprising:
a hollow cylindrical shaft wherein the apex is threaded on the outer surface and wherein a first groove runs the length of the inner surface;
a cylindrical blade attached to the base of said shaft wherein the cutting edge is the base of the blade and wherein a second groove runs the length of the outer surface and aligns with said first groove and wherein a hole through the blade is located in said second groove;
a wire configured as a lasso on the inside of said blade wherein said wire is flush with the inner surface of said blade and wherein said wire exits through said hole and wherein said wire runs along said second groove and continues along said first groove in the shaft and;
a removable handle wherein said wire is attached and;
a sleeve wherein threads on the inner surface are complementary with threads on said shaft and wherein the base of said shaft may be extended beyond the base of said sleeve.
